(19) United States
(12) Patent Application Publication
(10) Pub. No.: US 2022/0213534 A1
(43) Pub. Date: Jul. 7, 2022

(54) Digital Resolution Detection of miRNA with Single Base Selectivity by Photonic Resonator Absorption Microscopy

(71) Applicant: THE BOARD OF TRUSTEES OF THE UNIVERSITY OF ILLINOIS, Urbana, IL (US)

(72) Inventors: Brian T. CUNNINGHAM, Champaign, IL (US); Taylor D. CANADY, Champaign, IL (US); Nantao LI, Champaign, IL (US); Andrew M. SMITH, Savoy, IL (US); Yi LU, Champaign, IL (US); Hanyuan ZHANG, Champaign, IL (US)

(21) Appl. No.: 17/606,567

(22) PCT Filed: Apr. 27, 2020
(86) PCT No.: PCT/US2020/030055

§ 371 Date: Oct. 26, 2021

Related U.S. Application Data
(60) Provisional application No. 62/840,040, filed on Apr. 29, 2019.

(51) Int. Cl. C12Q 1/6837; C12Q 1/6825

(52) U.S. Cl. CPC C12Q 1/6837; C12Q 1/6825

(57) ABSTRACT

Assays using nanoparticle probes can be used to detect a target oligonucleotide with digital resolution by measuring the peak wavelengths and/or peak intensities of resonantly reflected light from locations on the surface of a photonic crystal (PC). The PC is functionalized with a capture oligonucleotide that binds to a nanoparticle probe that has bound to the target analyte. The binding of the nanoparticle probe to the PC shifts the peak wavelength and reduces the peak intensity of the resonantly reflected light at the binding location. An example nanoparticle probe includes a metallic nanoparticle conjugated to a probe oligonucleotide bound to a protector oligonucleotide. The probe oligonucleotide includes a first portion complementary to the target oligonucleotide and a second portion complementary to the capture oligonucleotide. The target oligonucleotide can bind to the probe oligonucleotide and displace the protector oligonucleotide, which exposes the second portion and enables binding to the capture oligonucleotide.

### CROSS-REFERENCE TO RELATED APPLICATIONS

This application claims priority to U.S. Provisional Patent Application No. 62/840,040, filed Apr. 29, 2019, which is incorporated herein by reference.

### STATEMENT REGARDING FEDERALLY SPONSORED RESEARCH

This invention was made with government support under 5R33CA177446-02 and 5R01GM086382-03 awarded by the National Institutes of Health. The Government has certain rights in this invention.

### BACKGROUND

The development of rapid and cost-effective diagnostics beneficially enables technologies for clinical applications in broad point of care settings. The prominent rise of liquid biopsy approaches to establish early disease detection, monitoring of treatments, prognostication and predicting pre-treatment outcomes further emphasizes the desirability of inexpensive high-performance assays. Among the numerous analytes in blood, circulating microRNA (miRNA) is an intriguing biomarker, with several studies correlating miRNA amount and variance to cancer type and metastatic state. However, the standard protocol of whole blood RNA isolation and purification prior to identification by quantitative reverse transcriptase PCR (qRT-PCR) is labor-intensive, requires amplification, and can suffer from target biases. In addition, microarray diagnostics exhibit low selectivity and limited dynamic range, and sequencing approaches involve elaborate sample processing, expensive equipment, long wait times, and bioinformatic expertise, all of which limit their point of care (POC) use. Electrochemical approaches are capable of ultrasensitive and amplification-free miRNA detection with a simple read out. However, developing a diagnostic that is both ultrasensitive and highly selective is desirable to effectively discriminate low concentrations of similar-sequence nucleic acids. Furthermore, a diagnostic assay that does not involve enzymatic amplification, pre-incubation, or washing is desirable for POC use.

### SUMMARY

Disclosed herein is a simple biosensor platform for miRNA that is capable of rapid digital signal accumulation with a wide dynamic range and highly selective single base mismatch discrimination using DNA nanotechnology. By tuning the probe-target reaction thermodynamics, highly selective nucleic acid detection is achievable, with single base discrimination. Moreover, energetically tuned DNA hybridization probes can recognize single base changes under large salinity, temperature, and concentration changes. Whereas DNA probes can be designed to be highly discriminatory towards nucleic acid variants, photonic crystal biosensors can achieve single particle resolution of bound nanoparticles. By combining the performance of selective DNA hybridization probes with digitally precise photonic crystal biosensors, it is possible to directly detect target miRNAs with single mismatch discrimination and low concentration sensitivity.

In one aspect, example embodiments provide an assay medium that can be used in methods and apparatus for detecting a target oligonucleotide. The assay medium comprises a buffer solution and a plurality of nanoparticle probes in the buffer solution. The nanoparticle probes comprise metallic nanoparticles in which each metallic nanoparticle is conjugated to a probe oligonucleotide with a protector oligonucleotide bound to the probe oligonucleotide. A first portion of the probe nucleotide is complementary to the target oligonucleotide such that the target oligonucleotide is able to bind to the probe oligonucleotide and displace the protector oligonucleotide therefrom. The assay medium further comprises an excess amount of the protector oligonucleotide in the buffer solution.

In another aspect, example embodiments provide a method that involves exposing a surface of a photonic crystal to (i) a sample comprising a target oligonucleotide, (ii) a plurality of nanoparticle probes configured to bind to the target oligonucleotide, wherein binding of the target oligonucleotide to a given nanoparticle probe displaces a protector oligonucleotide therefrom and enables the given nanoparticle probe to bind to the surface of the photonic crystal, and (iii) an excess amount of the protector oligonucleotide. The method further involves determining a number of nanoparticle probes that have bound to the surface of the photonic crystal and correlating the number of nanoparticle probes that have bound to the surface of the photonic crystal with an abundance of the target oligonucleotide.

In yet another aspect, example embodiments provide a method that involves providing a functionalized photonic crystal, wherein the functionalized photonic crystal comprises a plurality of probe oligonucleotides bound to a surface of the photonic crystal, wherein each probe oligonucleotide is bound to a protector oligonucleotide and includes a first portion that is complementary to a target oligonucleotide such that the target oligonucleotide is able to bind to the probe oligonucleotide and displace the protector oligonucleotide therefrom, and wherein the probe oligonucleotide further includes a second portion that is exposed when the target oligonucleotide displaces the protector oligonucleotide. The method also involves exposing the functionalized photonic crystal to (i) a sample comprising the target oligonucleotide and (ii) conjugated nanoparticles, wherein each conjugated nanoparticle comprises a metallic nanoparticle conjugated to a reporter oligonucleotide that is configured to bind to the second portion of the probe oligonucleotide so as to form an individual nanoparticle probe bound to the surface of the photonic crystal. The method further involves determining a number of nanoparticle probes bound to the surface of the photonic crystal and correlating the number of nanoparticle probes bound to the surface of the photonic crystal with an abundance of the target oligonucleotide in the sample.

### BRIEF DESCRIPTION OF THE DRAWINGS

FIGS. 1A-1F schematically illustrate an assay for miRNA-375, according to an example embodiment.

FIGS. 2A-2D illustrate aspects of a photonic crystal (PC), according to an example embodiment.

FIG. 3A schematically illustrates a nanoparticle probe, including a metallic nanoparticle, probe oligonucleotide, and protector oligonucleotide, before the nanoparticle probe has bound to the target oligonucleotide, according to an example embodiment.

FIG. 3B schematically illustrates the nanoparticle probe shown in FIG. 3A after an instance of the target oligonucleotide has bound to the probe oligonucleotide and displaced the protector oligonucleotide, according to an example embodiment.

FIG. 3C schematically illustrates the nanoparticle probe shown in FIG. 3B after the nanoparticle probe has been captured by a capture oligonucleotide conjugated to a surface of a PC, according to an example embodiment.

FIG. 4 illustrates the results of simulations showing a synergistic coupling between the surface plasmon resonance of a gold nanoparticle (AuNP) and the guided mode resonance of a PC, according to an example embodiment.

FIGS. 5A-5D illustrate aspects of AuNP—PC coupling behavior. FIG. 5A is an SEM image of four nanoparticle probes bound to a PC surface. FIG. 5B illustrates the near-field electric field intensity distribution of a gold nano-urchin on a PC surface according to finite difference time domain (FDTD) simulations. FIG. 5C illustrates reflectance spectra of the bare PC and AuNP—PC hybrid according to simulations. FIG. 5D illustrates an experimental greyscale image and corresponding 3D contour plot showing peak wavelength shifts caused by AuNPs bound to the PC surface.

FIG. 6A is a side sectional view of a liquid-containing compartment that includes a PC as its bottom surface, according to an example embodiment.

FIG. 6B is a top view of the PC shown in FIG. 6A after individual nanoparticle probes have bound to the surface of the PC, according to an example embodiment.

FIG. 7 is schematic illustration of an example photonic resonator absorption microscopy (PRAM) instrument, according to an example embodiment.

FIGS. 8A-8C show the results of experiments that were performed to detect miRNA-375 over a wide range of concentrations. FIG. 8A shows peak wavelength greyscale images of the PC surface taken at various times for each of the miRNA-375 concentrations. FIG. 8B is an expanded view of one of the greyscale images with arrows identifying representative instances of single nanoparticle probes bound to the surface of the PC. FIG. 8C is a graph showing the particle counts that were measured for a blank and for each of the miRNA-375 concentrations.

FIGS. 9A-9C show the results of experiments using single-nucleotide variants (SNVs) of the target miRNA-375. FIG. 9A shows peak wavelength greyscale images of the PC surface taken at 1 minute and 120 minutes for each of the SNVs. FIG. 9B is a graph showing the particle counts for each of the SNVs and for the target miRNA-375 at the same concentration. FIG. 9C is a graph showing the results of tuning the amount of excess protector oligonucleotide to increase the selectivity for the target miRNA-375 as compared to one of the SNVs (MM).

FIGS. 10A and 10B show the results of experiments using various concentrations of the target miRNA-375 in a much higher concentration of one of the SNVs (MM₅). FIG. 10A shows peak wavelength greyscale images of the PC surface taken at 1 minute and 120 minutes for 1 pM MM₅ with no miRNA-375 and for 1 pM MM₅ with each of the concentrations of miRNA-375. FIG. 10B is a graph showing the particle counts for each of the concentrations of miRNA-375.

FIGS. 11A-11G illustrate aspects of a processing and counting algorithm for PRAM-acquired images, according to an example embodiment.

FIGS. 12A, 12B, 12C, and 12D schematically illustrate steps in an assay for a target oligonucleotide, according to an example embodiment.

### DETAILED DESCRIPTION

### 1. Overview

Circulating exosomal miRNA represents a potentially useful class of blood-based biomarkers for cancer liquid biopsy. The detection of miRNAs at very low concentration and with single-base discrimination without using sophisticated equipment, large volumes, or elaborate sample processing is a challenge. To address this challenge, disclosed herein is an approach that is highly specific for a target miRNA sequence and has the ability to provide “digital” resolution of individual target molecules with high signal-to-noise ratio. In an example embodiment, gold nanoparticles are prepared with thermodynamically optimized nucleic acid “toehold” probe oligonucleotides that, when binding to a target miRNA sequence, displace a protector oligonucleotide and reveal a capture sequence that is used to selectively bind the target-probe-nanoparticle complex to a photonic crystal (PC) biosensor surface. By matching the surface plasmon resonant wavelength of the nanoparticle to the resonant wavelength of the PC, the reflected light intensity from the PC is dramatically and locally quenched by the presence of each individual nanoparticle, thereby enabling a type of biosensor microscopy that has been referred to as Photonic Resonator Absorption Microscopy (PRAM). Relevant aspects of PRAM are described in U.S. patent application Ser. No. 16/170,111, filed Oct. 25, 2018, which is incorporated herein by reference. Presented herein are experimental results that show that dynamic PRAM imaging of nanoparticle probes captured on the PC surface can provide direct 100 aM limit of detection and single-base mismatch selectivity in a 2-hour kinetic discrimination assay. These results demonstrate that ultrasensitivity and high selectivity can be achieved with direct read-out diagnostics.

A PC is a sub-wavelength grating structure consisting of a periodic arrangement of a low refractive index material coated with a high refractive index layer. When the PC is illuminated with a broadband light source, high order diffraction modes couple light into and out of the high index layer, destructively interfering with the zeroth-order transmitted light. At a particular resonant wavelength and incident angle, complete interference occurs and no light is transmitted, resulting in nearly 100% reflection efficiency. Various aspects of photonic crystal biosensors are described in U.S. Pat. Nos. 7,479,404, 7,521,769, 7,531,786, 7,737,392, 7,742,662, and 7,968,836, which patents are incorporated herein by reference.

When a material is adsorbed on the surface of a PC, the resonant reflection from the PC can be affected in two ways. First, the adsorbed material can cause a shift in the resonant Peak Wavelength Value (PWV), typically shifting the PWV to longer wavelengths. Second, the adsorbed material can cause a reduction in the Peak Intensity Value (PIV) at the resonant wavelength. Both of these effects, the shift in the PWV and the reduction in the PIV are highly localized. Thus, different locations on the surface of the PC may have different PWVs and different PIVs, depending on the materials at those locations.

Two mechanisms exist for locally reducing the reflected intensity from a PC at the resonant wavelength: absorption and scattering. The absorption mechanism is believed to work in the following manner. A substance that possesses optical absorption at the resonant wavelength of the PC will locally reduce the intensity of the resonant wavelength due to a mechanism through which the attached material (i.e., within the evanescent field region on the surface of the PC) gathers energy into itself, where it is dissipated by heating the surrounding environment. If the reflected intensity from the PC is observed at the PC resonant wavelength, one would observe a “hole” in the reflected intensity at the location of the optical absorber. Typical absorbing materials may include metals (e.g., Au, Ag, Pd), semiconductors, quantum dots, or colored polymers. Alternatively, a material attached to the PC surface that is not an optical absorber may cause a localized reduction in the intensity of the resonant wavelength if the material has sufficient dielectric permittivity contrast to its surrounding environment to outcouple light from the PC by scattering.

Disclosed herein are approaches that make use of PRAM imaging to detect a target analyte in a sample. In example embodiments, the target analyte could be an oligonucleotide, such as a micro RNA (miRNA), messenger RNA (mRNA), splice variant RNA, or circulating DNA. Alternatively, the target analyte could be a protein, an exosome, or a viral particle. In a first approach, a nanoparticle probe binds to the target analyte and then binds to the surface of a PC. In a second approach, a probe bound to the surface of the PC binds to the target analyte and then conjugated nanoparticles bind to the target-activate probes to form nanoparticle probes bound to the surface of the PC. In either approach, PRAM imaging can be used to identify individual nanoparticle probes bound to the surface of the PC, based on localized shifts in PWV and/or localized reductions in PIV that are caused by the nanoparticle probes having an absorption (e.g., a surface plasmon resonance) at a wavelength that corresponds to the PC's resonant wavelength. The number of individual nanoparticle probes bound to the PC that are identified in this way can be counted and correlated to an abundance of the target analyte in the sample.

In the first approach, the nanoparticle probe can be a metallic nanoparticle that is functionalized to bind to the target analyte. For example, to bind to a target oligonucleotide, the metallic nanoparticle can be functionalized with a probe oligonucleotide in which at least a portion of the probe oligonucleotide is complementary to the target oligonucleotide. To bind to other types of target analytes (e.g., a target protein, exosome, or viral particle), the metallic nanoparticle can be functionalized with an antibody, aptamer, protein, or other molecule that specifically binds to the target analyte.

The nanoparticle probe can also be designed such that it can bind to a functionalized PC surface after binding to the target analyte. The PC surface can be functionalized with a capture oligonucleotide, capture antibody, or capture protein. For example, if the target analyte is a target oligonucleotide, the nanoparticle probe can include a probe oligonucleotide that has a first portion that is complementary to the target oligonucleotide, and the PC surface can be functionalized with a capture oligonucleotide that is complementary to a second portion of the probe oligonucleotide. Initially, the nanoparticle probe can include a protector oligonucleotide that is bound to at least part of the first portion of the probe oligonucleotide and at least part of the second portion of the probe oligonucleotide. The target oligonucleotide can bind to the probe oligonucleotide and displace the protector oligonucleotide from the probe oligonucleotide. This, in turn, exposes the second portion of the probe oligonucleotide such that the capture oligonucleotide is able to bind to the probe oligonucleotide. In this way, the nanoparticle probe can bind to the PC surface via the capture oligonucleotide after first binding to the target oligonucleotide.

If the target analyte is a target protein, the nanoparticle probe can include a probe molecule (e.g., an antibody, aptamer, or protein) that specifically binds to a first epitope on the target protein, and the PC surface can be functionalized with a secondary antibody that specifically binds to a second epitope on the target protein. In this way, the nanoparticle probe can bind to the PC surface via the secondary antibody after first binding to the target protein.

If the target analyte is a target exosome or viral particle, the nanoparticle probe can include a probe molecule (e.g., a protein or antibody) that specifically binds to a protein at a first location on the outer surface of the exosome or viral particle, and the PC surface can be functionalized with a capture molecule (e.g., a protein or antibody) that specifically binds to a protein at a second location on the outer surface of the exosome or viral particle. In this way, the nanoparticle probe can bind to the PC surface via the capture molecule after first binding to the target exosome or viral particle. In some implementations, the capture molecule and the probe molecule could bind to the same protein that is expressed at different locations at the outer surface of the exosome or viral particle. Thus, the capture molecule on the PC surface could be the same as the probe molecule in the nanoparticle probe.

To perform an assay for a target analyte in a sample, the sample can be mixed with a buffer solution that includes nanoparticle probes that can bind to the target analyte, and the mixture can be exposed to a PC surface that is functionalized to bind to the nanoparticle probes that have bound to the target analyte. The PC surface can then be imaged using an instrument that can detect localized shifts in PWV and/or localized reductions in PIV caused by the nanoparticle probes binding to the PC surface. Individual instances of bound nanoparticle probes can be counted in the images, and the number of bound nanoparticle probes can be correlated to an abundance of the target analyte in the sample.

In the second approach, a probe that is designed to bind to a target analyte is initially bound to the PC surface. The probe could include an oligonucleotide, antibody, aptamer, or protein that specifically binds to the target analyte. To perform an assay for a target analyte in a sample, the functionalized PC surface is exposed to the sample so that individual instances of the target analyte can bind to individual probes. The functionalized PC surface is then exposed to conjugated nanoparticles. When a probe has bound to the target analyte, the target-activated probe can bind to a conjugated nanoparticle to form a nanoparticle probe that is bound to the PC surface. The PC surface can then be imaged as described above. Individual instances of bound nanoparticle probes can be counted in the images, and the number of bound nanoparticle probes can be correlated to an abundance of the target analyte in the sample.

A sample could also be tested for multiple, different target analytes in a multiplexed assay. For example, the sample could be mixed with a buffer solution that includes multiple different types of nanoparticles probes, with each type of nanoparticle probe being able to bind to a different target analyte that may be present in the sample. The PC surface, in turn, can include multiple different regions, with different regions being functionalized to bind to different types of nanoparticle probes. The multiple different regions of the PC surface can be imaged to detect localized shifts in PWV and/or localized reductions in PIV. In this way, the nanoparticle probes that bind to the PC surface in each respective region can be counted and correlated to an abundance of the corresponding target analyte in the sample. In this approach, the PC surface with the multiple different regions could be provided at the bottom surface of a liquid-containing compartment in which the sample is mixed with the multiple different types of nanoparticles probes. Alternatively, a sample could be tested for multiple, different target analytes by dividing the sample into separate volumes. Each separate sample volume could then be mixed with buffer solution containing nanoparticle probes that can bind to one particular target analyte, and the resulting mixture can then be exposed to a PC surface that is functionalized to bind to the nanoparticle probes that have bound to that particular target analyte.

In some implementations, the buffer solution that is mixed with the sample can include components that can increase the selectivity of the nanoparticle probes binding to the target analyte. For example, if the target analyte is a target oligonucleotide, it may be desirable to increase the selectivity of the probe oligonucleotide in the nanoparticle probe for the target oligonucleotide over single-nucleotide variants of the target oligonucleotide. In one approach for increasing selectivity, the buffer solution can include one or more sink-probes that are complementary to one or more of the single-nucleotide variants. In another approach, the buffer solution can include an excess amount of the protector oligonucleotide so as to make binding of the target oligonucleotide to the probe oligonucleotide (with associated displacement of the protector oligonucleotide) more thermodynamically favorable as compared to one or more competitive binding reactions (e.g., binding of one more single-nucleotide variants to the probe oligonucleotide with associated displacement of the protector oligonucleotide). For example, the binding of the target oligonucleotide with displacement of the protector oligonucleotide could be associated with a Gibbs free energy of reaction, ΔG_{TO}, and binding of a single-nucleotide variant with displacement of the protector oligonucleotide could be associated with a Gibbs free energy of reaction, ΔG_{SNV}. The amount of excess protector oligonucleotide in the buffer solution could be selected such that ΔG_{TO} is either zero or negative and ΔG_{SNV} is positive.

### 2. Example Assay Format

In example embodiments, nanoparticle probes are used in an assay to detect a miRNA as a target oligonucleotide. The components of the assay may include (i) the nanoparticle probes, (ii) a sample containing the miRNA, (iii) excess protector oligonucleotide, (iv) a PC surface that has been functionalized with a capture oligonucleotide, and (v) buffer solution.

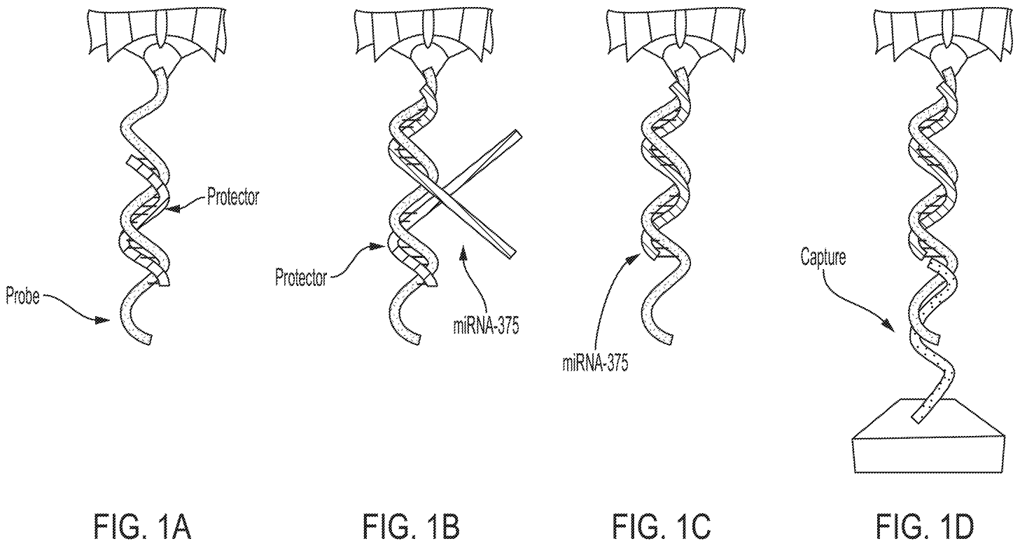

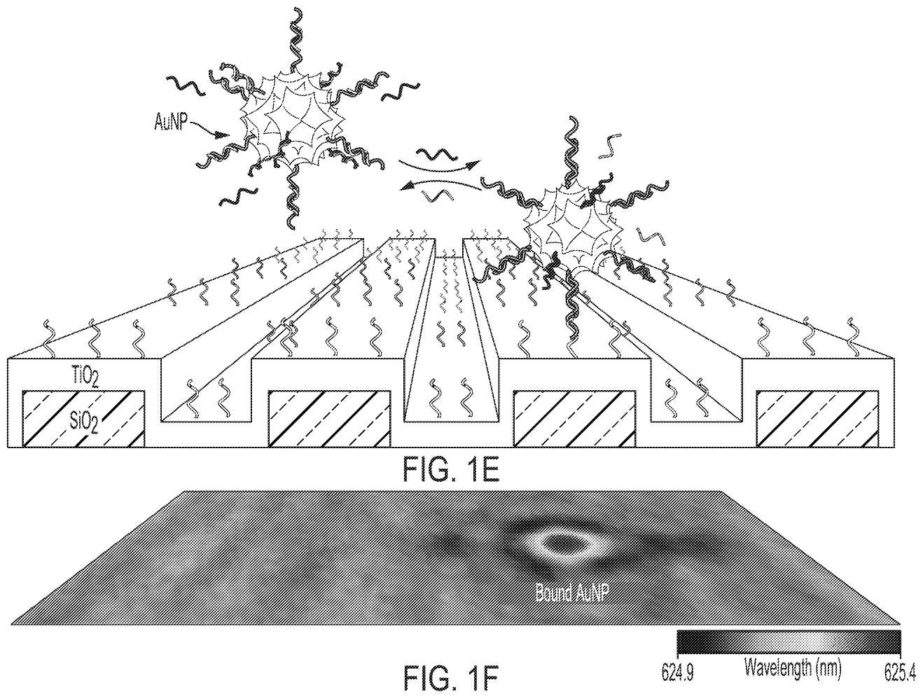

FIGS. 1A-1F schematically illustrate an example assay. In this example, miRNA-375 is the target oligonucleotide. As shown in FIG. 1A, each nanoparticle probe includes a metallic nanoparticle functionalized with a probe oligonucleotide and with a protector oligonucleotide bound to the probe oligonucleotide.

The metallic nanoparticle in this example is a gold nano-urchin that is about 100 nm in diameter. The gold nano-urchin has a surface plasmon resonance at a wavelength (approximately 625 nm) that matches the resonance wavelength of the PC, so that binding of the nanoparticle on the PC surface is associated with a shift in PWV and reduction in PIV. It is believed that the spiked surface of the nano-urchin enhances these effects. It is to be understood, however, that other metals and/or shapes could be used. For example, other metals (e.g., silver) may have a surface plasmon resonance at a wavelength that matches the resonance wavelength of the PC. Further, the metallic nanoparticle may include a magnetic material, such as iron or nickel, in addition to the gold, silver, or other metal with the appropriate surface plasmon resonance, so as to allow for magnetic manipulation of the nanoparticle probes. For example, a magnetic field may be used to attract magnetic nanoparticle probes to the PC surface so as to accelerate the process of binding nanoparticle probes to the PC surface (the binding process may otherwise rely on nanoparticle probes reaching the PC surface by diffusion). Alternatively or additionally, a magnetic field may be used to repel magnetic nanoparticle probes to the PC surface (e.g., to push away nanoparticle probes that have landed on the PC surface by gravity but have not bound to the target oligonucleotide and so have not biochemically bound to the capture oligonucleotide on the PC surface). The metallic nanoparticle could also have different shapes, such as a spherical or near-spherical shape that provides a smooth outer surface or a shape with a non-spherical symmetry (e.g., a rod shape).

As shown in FIG. 1B, the target oligonucleotide (miRNA-375) can bind to the probe oligonucleotide in the nanoparticle probe, and this binding reaction also displaces the protector oligonucleotide. The nanoparticle probe once the target oligonucleotide (miRNA-375) has bound to the probe oligonucleotide and the protector oligonucleotide has been displaced is shown in FIG. 1C. The displacement of the protector oligonucleotide exposes a portion of the probe oligonucleotide that is complementary to the capture oligonucleotide on the PC surface. Thus, the probe oligonucleotide in the nanoparticle probe can bind to the capture oligonucleotide on the PC surface, as shown in FIG. 1D, once the target oligonucleotide has bound to the probe oligonucleotide and displaced the protector oligonucleotide.

As shown in FIG. 1E, nanoparticle probes that have not bound to the target oligonucleotide (such as the AuNP shown on the left) are not able to bind to the capture oligonucleotide on the PC surface, whereas nanoparticle probes that have bound to one or more instances of the target oligonucleotide (such as the AuNP shown on the right) are able to bind to the capture oligonucleotide on the PC surface. Further, when a nanoparticle probe binds to the PC surface via the capture oligonucleotide, the PWV (resonance wavelength of the PC) shifts at that location, as illustrated in FIG. 1F. For example, areas of the PC surface where nanoparticle probes have not bound may have a PWV of about 624.9 nm, whereas the PWV at or near the location where a nanoparticle probe has bound will be shifted to a longer wavelength (e.g., shifted to 625.4 nm).

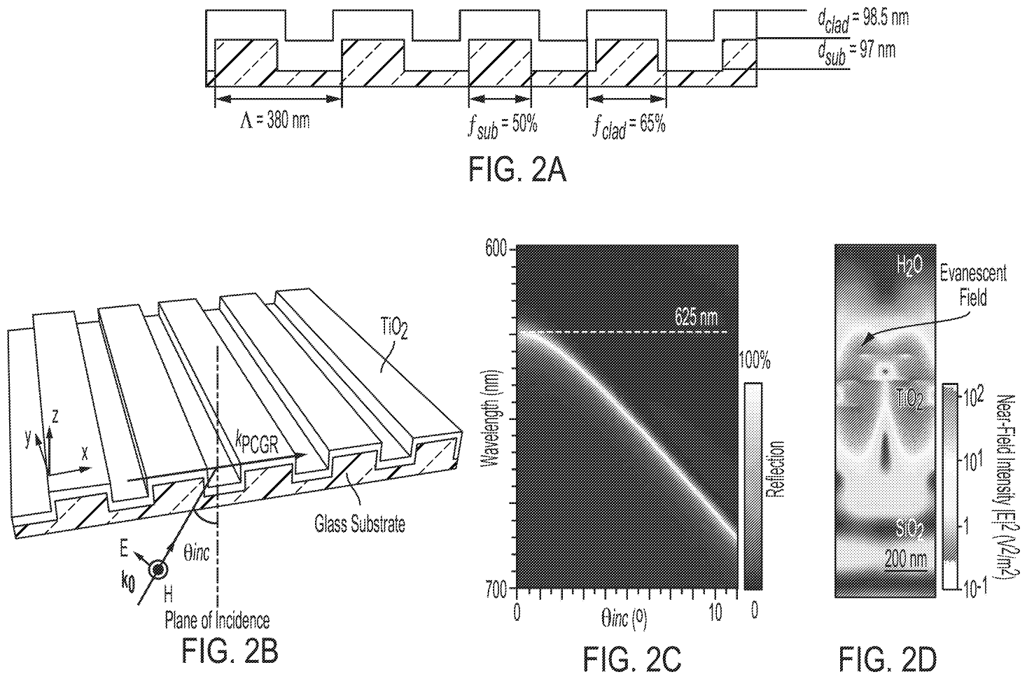

FIGS. 2A-2D illustrate aspects of an example photonic crystal (PC) that may be used. The PC is a subwavelength periodic grating structure which is highly sensitive to the presence of plasmonic nanoparticle surface binding in its evanescent field when the photonic crystal resonance wavelength and the plasmonic nanoparticle resonance are matched. In this example, the PC comprises a high refractive index cladding material (TiO₂; n=2.4) that is formed on a substrate of a lower refractive index material (SiO₂; n=1.5) that is structured as a one-dimensional grating on a glass support. Shown in FIG. 2A is a schematic cross-sectional view of the PC that includes dimensions of the cladding, dimensions of the substrate, and the period of the grating structure (Λ=380 nm). The diagram of FIG. 2B illustrates a transverse magnetic (TM) polarized excitation caused by incident light at a particular angle of incidence, θ_{inc}. The optical resonance can be spectrally tuned by altering the angle of incidence. This is illustrated in FIG. 2C, which shows the far-field reflectance spectrum of the bare PC as a function of θ_{inc}, according to simulations. Further, FIG. 2D shows results from finite difference time domain (FDTD) calculations of the electric field amplitude distribution within one period of the PC under normal incidence and the 625 nm wavelength illustrated in FIG. 2C.

Table 1 below identifies the nucleotide sequences for the target oligonucleotide (miRNA-375) and for five different single-nucleotide variants (single-nucleotide polymorphisms), along with the nucleotide sequences for the probe oligonucleotide, protector oligonucleotide, and capture oligonucleotide that were used in the studies reported herein.

TABLE 1

| DNA/RNA | Sequence (5′-3′) |
|---|---|
| Target Oligonucleotide | UUU GUU CGU UCG GCU CGC GUG A |
| (miRNA-375) | |
| 1^{st} Nucleotide Mutation (MM₁) | CUU GUU CGU UCG GCU CGC GUG A |
| 5^{th} Nucleotide Mutation (MM₅) | UUU GAU CGU UCG GCU CGC GUG A |
| 12^{th} Nucleotide Mutation (MM₁₂) | UUU GUU CGU UCC GCU CGC GUG A |
| 18^{th} Nucleotide Mutation (MM₁₈) | UUU GUU CGU UCG GCU CGA GUG A |
| 22^{nd} Nucleotide Mutation (MM₂₂) | UUU GUU CGU UCG GCU CGC GUG C |
| Probe Oligonucleotide | CCC ACC TAC ATC ACG CGA GCC GAA |
| | CGA ACA AAA AAA/3DTPA/ |
| Protector Oligonucleotide | GTT CGG CTC GCG TGA TGT AGG |
| Capture Oligonucleotide | TGT AGG TGG G/3AmMO/ |

The probe oligonucleotide is functionalized with a dithiol group on the 3′-end, as denoted by /3DTPA/, for attachment to the metallic nanoparticle. The capture oligonucleotide is functionalized with an amino group on the 3′-end, as denoted by /3AmMO/, for attachment to the PC surface.

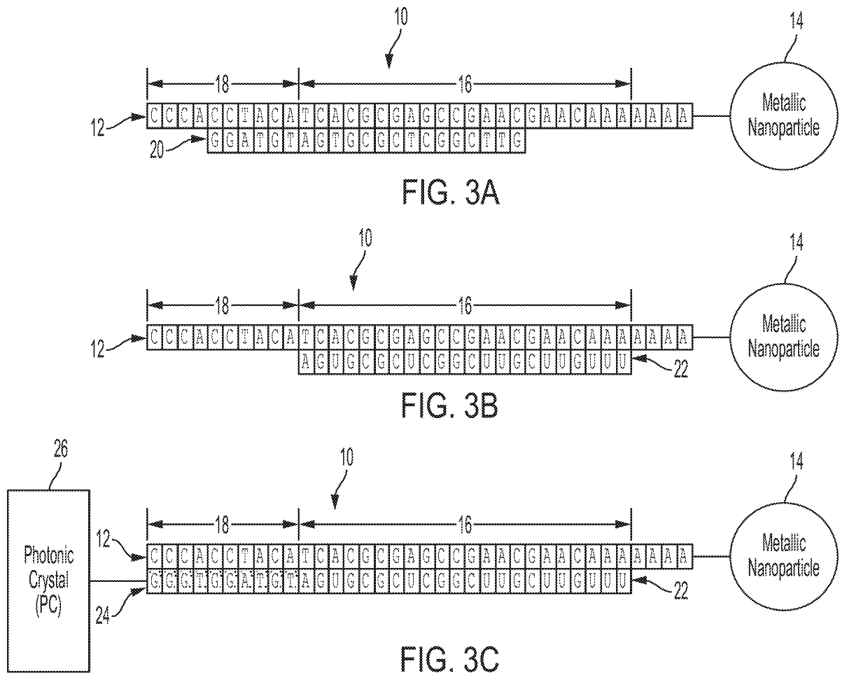

FIGS. 3A-3C schematically illustrate a nanoparticle probe 10 that includes the probe oligonucleotide and protector oligonucleotide identified above in Table 1 (as shown in FIG. 3A), that binds to the target oligonucleotide (miRNA-375) identified in Table 1 (as shown in FIG. 3B), and then binds to the capture oligonucleotide identified above in Table 1 (as shown in FIG. 3C).

FIG. 3A shows the nanoparticle probe 10 before it has bound to the target oligonucleotide. As shown, the nanoparticle probe 10 includes the probe oligonucleotide 12 conjugated to a metallic nanoparticle 14. The probe oligonucleotide 12 includes a first portion 16 that is complementary to the target oligonucleotide and a second portion 18 that is complementary to the capture oligonucleotide. The nanoparticle probe 10 further includes the protector oligonucleotide 20 bound to the probe oligonucleotide 12 so as to cover part of the first portion 16 and part of the second portion 18.

FIG. 3B shows the nanoparticle probe 10 after it has bound to an instance of the target oligonucleotide 22. As shown, the target oligonucleotide 22 is bound to the first portion 16 of the probe oligonucleotide 12. However, since the protector oligonucleotide 20 has been displaced, the second portion 18 of the probe oligonucleotide 12 is exposed. FIG. 3C shows the nanoparticle probe 10 after it has been captured by an instance of the capture oligonucleotide 24 conjugated to the surface of a photonic crystal (PC) 26. Specifically, the capture oligonucleotide 24 (indicated by shading) binds to the second portion 18 of the probe oligonucleotide 12.

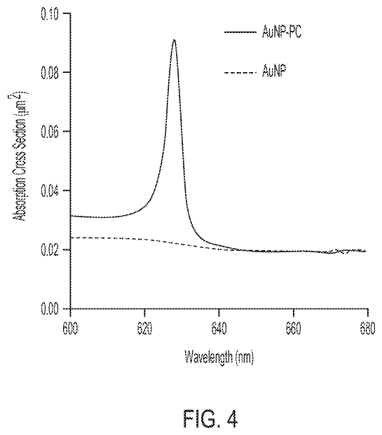

By matching the AuNP surface plasmon resonance to the PC guided resonance (PCGR) wavelength, the synergistic coupling between the two resonators results in a drastically enhanced AuNP absorption cross section. FIG. 4 illustrates the results of simulations that illustrate this effect. The bare AuNP has a broadband plasmonic absorption peak with a central wavelength at 607 nm. The presence of the PCGR significantly enhances the absorption cross section at the resonance wavelength (628 nm).

In addition, the AuNPs can include a protruding tip morphology (e.g., the gold nanoparticles could be gold nano-urchins), which beneficially allows for improved light harvesting across the particle surface. Moreover, gold nano-urchins have been found to provide an isotropic enhancement, in contrast to gold nanorods, which have an orientation-dependent enhancement upon PC binding.

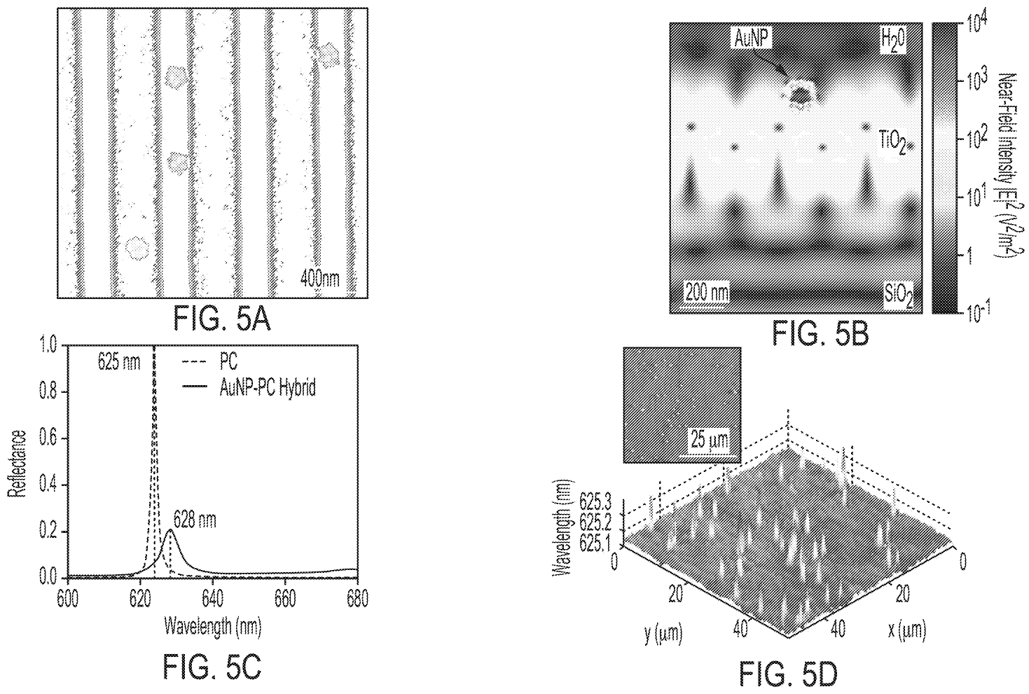

FIGS. 5A-5D illustrates aspects of AuNP—PC coupling behavior. A scanning electron microscope (SEM) image of four nanoparticle probes comprising gold nano-urchins (100 nm diameter) bound to the PC surface is shown in FIG. 5A. The near-field electric field intensity distribution of a gold nano-urchin on a PC surface according to FDTD simulations is shown in FIG. 5B. Those simulations demonstrate a field enhancement of about 10⁴ at the AuNP sharp tip features and is shown to be sensitive to the incident angle and wavelength. Additional simulations were used to calculate the reflectance spectrum of the PC alone and the AuNP—PC hybrid, as shown in FIG. 5C. According to this simulation, formation of the AuNP—PC hybrid shifts the peak reflectance wavelength from 625 nm to 628 nm (Δλ) and reduces the reflectance peak intensity (ΔI), due to coupling between the surface plasmon resonance of the AuNP and the PCGR. Moreover, these effects are localized at the site where the AuNP is attached to the PC surface. Photonic Resonator Absorption Microscopy (PRAM) can be used to observe the peak reflectance wavelength shift (Δλ) and reduction in reflectance peak intensity (ΔI) associated with each individual surface-attached AuNP. This is shown in FIG. 5D. The experimental 2D grey scale PRAM image (top left) is represented in the 3D contour plot demonstrating the individual gold nanoparticle peak wavelength shifts.

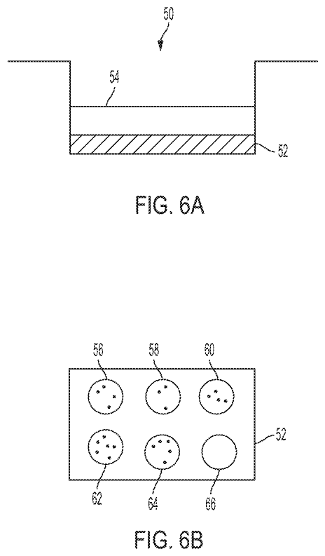

A multiplexed assay format can also be used to detect multiple target analytes in a sample. FIGS. 6A and 6B illustrate an example of how such a multiplexed assay could be implemented. In this example, the sample is tested for six different target oligonucleotides (e.g., six different miRNAs) using six different types of nanoparticle probes, with each type of nanoparticle probe including a probe oligonucleotide that specifically binds to one of the target oligonucleotides. FIG. 6A is a side sectional view of a liquid-containing compartment 50 that has a PC 52 as its bottom surface. The sample and buffer solution containing the six different types of nanoparticle probes are added to the liquid-containing compartment 50 to form a liquid mixture 54 on the PC 52. FIG. 6B is a top view of the PC 52 after the nanoparticle probes in the liquid mixture 54 have had an opportunity to first bind to the target oligonucleotides in the sample and then bind to the surface of the PC 52. As shown in FIG. 6B, the surface of the PC 52 is functionalized with six different capture oligonucleotides in six different capture regions 56, 58, 60, 62, 64, and 66. The capture oligonucleotides in each capture region specifically bind to the probe oligonucleotide in one of the six different types of nanoparticle probes. In this way, each capture region can specifically capture nanoparticle probes that have bound to one of the target oligonucleotides. In the example shown in FIG. 6B, capture region 56 has captured four nanoparticle probes that are bound to target 1, capture region 58 has captured three nanoparticle probes that are bound to target 2, capture region 60 has captured four nanoparticle probes that are bound to target 3, capture region 62 has captured six nanoparticle probes that are bound to target 4, and capture region 64 has captured five nanoparticle probes that are bound to target 5. Further, while capture region 66 is configured to capture nanoparticle probes that are bound to target 6, capture region 66 has not captured any nanoparticle probes in this example. PRAM imaging can be used to count the number of nanoparticle probes captured in each of the capture regions 56-66, and the number of each nanoparticle probe in each capture region can be correlated to an abundance of the corresponding target oligonucleotide in the sample.

### 3. Example PRAM Instrument

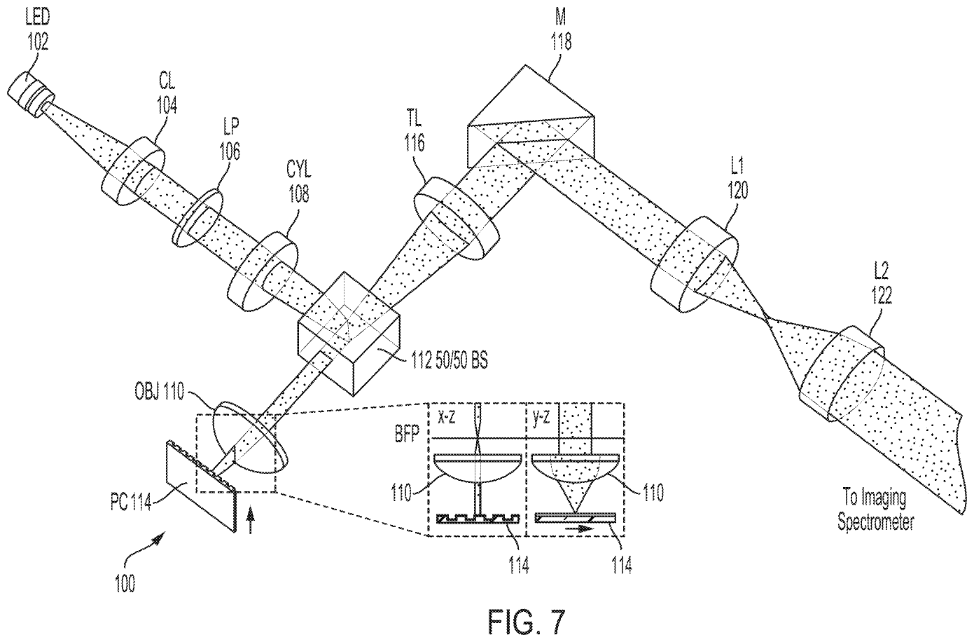

FIG. 7 is a schematic illustration of an example photonic resonator absorption microscopy (PRAM) instrument 100. The main body of the instrument is based on a commercially available inverted microscope (Carl Zeiss Axio Observer Z1). A broadband fiber-coupled LED 102 (Thorlabs M625F2, output power 15 mW) is used as the illumination light source. This light source has a nominal wavelength of 625 nm and a bandwidth (FWHM) of 15 nm. After passing through a collimating lens 104 (Thorlabs F810SMA-635), the light beam is linearly polarized by a linear polarizer 106 to provide a beam of TM-polarized illumination. A cylindrical lens 108 (Thorlabs U4667RM-A, f=200 mm) then focuses the beam onto the back focal plane of an objective lens 110 (Zeiss LD Plan-Neofluar 40×/0.6 NA) via a 50/50 beamsplitter 112. The objective lens 110 focuses the illumination onto the surface of a PC 114 in a plane parallel to the grating structure (the y-z plane shown in the right inset) such that the illumination is collimated by the cylindrical lens 108 in a plane transverse to the grating structure (the x-z plane shown in the left inset). Under normal incidence, the width of the focused light beam on the sample plane is about 1 μm. The reflected light from the sample on the PC 114 contains the resonant reflected spectrum from which the PWV and PIV can be determined.

The reflected light passes through the 50/50 beam splitter 112 and a tube lens 116 to reach a mirror 118. The mirror 118 reflects the light onto a relay lens group comprising lenses 120 and 122. The relay lens group provides 3× magnification and serves as the coupler between the inverted microscope and an imaging spectrometer (Princeton Instrument, Acton SpectraPro-2500i). Within the spectrometer, the reflected light from the sample is dispersed by the diffraction grating and focused onto a CCD camera mounted at the exit port. The CCD camera can obtain an image that includes a spatially resolved spectrum for each point of the illuminated line on PC 114. To acquire a continuous image over a certain region on the PC surface, a motorized sample stage (Applied Scientific Instruments, MS2000) translates the PC 114 along the y-axis with an increment of 0.15 μm, while the CCD camera synchronously captures the reflection spectra of each imaged line.

An analysis system coupled to the CCD camera can generate PWV and PIV images based on the data from the CCD camera. The analysis system may also control the motorized stage and CCD camera. The analysis system could be, for example, a computing device that is programmed with software for analyzing the images acquired by the CCD camera, to determine peak wavelengths and intensities of resonantly reflected wavelengths and to generate PWV and PIV images. The analysis system could also be programmed to analyze the images for digital assays. Such analysis by the analysis system could involve identifying areas in a PIV image that have reduced PIV and counting the number of particles bound to the surface of a PC based on the number of reduced-PIV areas in the PIV image. The analysis system could also correlate the number of particles bound to the surface of the PC with an abundance of an analyte in the sample, for example, based on each bound particle being bound to one instance of the analyte. Thus, the analysis system may include a processor and non-transitory data storage that stores instructions that are executable by the processor to perform any of the functions described herein.

### 4. Detection of miRNA-375 Using PRAM

The PRAM instrument shown in FIG. 7 and described above was used to study the prostate cancer biomarker miRNA-375. The studies used nanoparticle probes comprising gold nano-urchins (100 nm diameter) that were conjugated to the probe oligonucleotide with protector oligonucleotide bound thereto, using the probe oligonucleotide, protector oligonucleotide, and capture nucleotide listed above in Table 1.

a. PC Fabrication

The PCs used in these studies were formed on 200 mm diameter glass wafers purchased from Corning (0.7 mm Eagle XG display grade glass). A 10 nm etch stop layer of Al₂O₃ was deposited on the glass wafer. The periodic grating structures were fabricated by depositing a layer of SiO₂ followed by large-area ultraviolet interference lithography performed by a manufacturer (Moxtek, Orem, Utah). The etched wafers were then coated with a cladding layer of TiO₂. Finally, the wafers were diced into 1 inch×3 inch chips.

b. Nucleic Acids

The oligonucleotides used in the experiments were purchased from Integrated DNA Technologies (Coralville, Iowa). The probe sequence is functionalized with a dithiol group on the 3′-end, followed by high-performance liquid chromatography purification. The capture oligonucleotide includes a 3′-amine for PC surface functionalization. The sequences of the oligonucleotides are shown above in Table 1.

c. Nanoparticle Probes

A maleimide gold NanoUrchin conjugation kit (Cytodiagnostics, Burlington, Ontario) was used for synthesizing the nanoparticle probes. A 50:1 (Probe:AuNP) stoichiometry was used during the conjugation ([AuNP]=1.3×10⁻¹⁰ M). A 50 μL volume of dithiol modified probe oligonucleotide (100 μM) was reduced in 5 mM of dithiothreitol (Sigma-Aldrich) made up in 1×TE buffer (Sigma-Aldrich), followed by 4 times extraction using ethyl acetate (Sigma-Aldrich). The extracted probe oligonucleotide solution was redispersed using kit-provided reaction buffer (90 μL) so that the final concentration was 6.4 nM. The probe was added to the lyophilized maleimide gold NanoUrchin, and incubated for one hour at room temperature while mixing gently in a rotator to ensure sufficient reaction of AuNPs to probe oligonucleotides. 10 μL of the kit-provided quencher solution was added to the mixture and incubated for another 15 minutes. The conjugated AuNPs were separated from the supernatant by 30 minutes of centrifugation at 300 g and then dispensed with 100 μL of 1×TE buffer solution containing 12.5 mM MgCl₂ (Sigma-Aldrich) and 0.025% TWEEN-20 (Sigma-Aldrich).

The conjugated AuNP solution was annealed to a stoichiometric amount of protector oligonucleotide, with the desired energetic tuning determining the exact ratio. The oligonucleotides were annealed at 80° C. for 2 mins at cooled 0.5° C. every 30 seconds to 18° C. (Eppendorf 5331 MasterCycler Gradient Thermal Cycler). The final protector-annealed DNA-AuNP product was stored at 4° C. until use.

d. PC Surface Functionalization

PC chips were sonicated in acetone (Sigma-Aldrich), isopropyl alcohol (Sigma-Aldrich), and deionized water respectively for 2 minutes and dried under a stream of compressed nitrogen, followed by a 200 W oxygen plasma treatment at a pressure of 500 mTorr for 10 minutes using a Pico Plasma System (Diener electronic, Germany). In a glass reaction chamber, (3-Glycidoxypropyl)trimethoxysilane (GLYMO, Gelest, Morrisville, Pa.) was vapor-deposited on the PC surface in a vacuum oven at a temperature of 80° C. under 30 Torr for at least 3 hours. For each PC chip in vapor-deposition, 100 μL of GLYMO was added in the containing glass reaction chamber. The deposited PC chips were removed from the oven and sonicated in toluene (Sigma-Aldrich), methanol (Sigma-Aldrich) and deionized water respectively for 2 minutes, and nitrogen dried. For the DNA functionalization of a 1 cm PC surface, a volume of 20 μL amino-terminated capture oligonucleotide dispersion in 1×TE buffer was redispersed into 180 μL of 1×TE buffer containing 0.05% TWEEN-20 (pH=9.0), and dispensed on the GLYMO-deposited PC surface. After 8 hours of incubation at room temperature, the PC chips were rinsed by a gradual decrease of TE buffer concentration from 1× to 0.01×. The final PC chips were sealed in a Petri dish container and stored at 4° C. until use. Immediately before use, SuperBlock (in TBS) blocking buffer (ThermoFisher Scientific) was added for 5 minutes and washed using 1×TE buffer.

e. Image Acquisition and Analysis

A line-scanning spectromicroscope was programmed to acquire the resonant reflectance spectrum of each pixel within the field-of-view. Upon the acquisition of the resonant reflection spectral peak of each pixel within field of view, a spectral deconvolution algorithm is applied to extract the resonant wavelength and the corresponding reflection intensity at location on the PC surface. Each reflection spectrum is deconvoluted into two Lorentzians: one Lorentzian centered at the main wavelength of the LED light source, representing the contribution from the LED light source; and another Lorentzian with a central wavelength that is a variable to be fitted, representing the reflected signal from the sample. Depending on which feature is in use, two imaging modalities can be obtained from one single image acquisition: peak intensity value (PIV) image and peak wavelength value (PWV) image, each representing the absorption efficiency and the resonance condition of each pixel within the sampled region. For PIV images, the height of the PC resonance peak in the reflection spectrum was attributed to each pixel in question, and vice versa for PWV images. Finally, a notch filter mask in the Fourier space is applied to remove nonuniformity in the background caused by line-scanning.

Nanoparticles in the PWV image are detected based on binarization of the image. In this approach, the acquired PWV images are first converted into grey scale images. The contrast is then adjusted based on a Contrast Limited Adaptive Histogram Equalization (CLAHE) algorithm, with a contrast enhancement limit set as 0.5. Next, the noise in the images is suppressed using a 2D Wiener filter using neighborhoods of size 5-by-5 to estimate the local image mean and standard deviation. Then, the filtered images are binarized with a threshold of 0.5, followed by erosion and dilation to remove background noise and holes in the images. The presence of nanoparticles results in a red shift in the peak wavelength value. Therefore, the binarization method can provide the total amount of nanoparticles presented in the image by simply summing all the individually partitioned “true” patterns. The local maxima within each segmented pattern are used as indicators of individual nanoparticle, and a watershed algorithm is applied to all the recognized patterns before the summation in order to avoid the inaccuracy caused by clustered nanoparticles.

f. Reaction Thermodynamics

The probe oligonucleotide and protector oligonucleotide were developed (using the webtool NUPACK) such that the reaction of miRNA-375 binding to the probe oligonucleotide with associated displacement of the protector oligonucleotide was more thermodynamically favorable than for competing reactions involving MM₁, MM₅, MM₂, MM₁₈, and MM₂₂, as summarized below in Table 2.

TABLE 2

| | Standard Free Energy G° |
|---|---|
| Hybridization Structure | (kcal/mol) |
| Protector-Probe (PC) | −36.06 |
| miRNA375-Probe (TC) | −37.42 |
| NM₁-Probe (MM₁C) | −36.43 |
| MM₅-Probe (MM₅C) | −33.85 |
| MM₁₂-Probe (MM₁₂C) | −31.71 |
| MM₁₈-Probe (MM₁₈C) | −32.80 |
| MM₂₂-Probe (MM₂₂C) | −36.43 |
| | Standard Free Energy of |
| Binding/Displacement Reaction | Reaction ΔG° (kcal/mol) |
| T + PC [symbol] TC + HP | −1.36 |
| MM₁ + PC [symbol] MM₁C + P | −0.37 |
| MM₅ + PC [symbol] MM₅C + P | 2.21 |
| MM₁₂ + PC [symbol] MM₁₂C + P | 4.35 |
| MM₁₈ + PC [symbol] MM₁₈C + P | 3.26 |
| MM₂₂ + PC [symbol] MM₂₂C + P | −0.37 |

The upper portion of Table 2 lists the standard Gibbs free energy G° of each hybridized structure obtained using the webtool NUPACK, assuming a temperature of 25° C. and a buffer solution containing 0.05 M Na⁺ and 0.0125 M Mg²⁺. In Table 2, the probe oligonucleotide is abbreviated as C, the protector oligonucleotide is abbreviated as P, the miRNA-375 target oligonucleotide is abbreviated as T, and MM₁, MM₅, MM₁₂, MM₁₈, and MM₂₂ are the single-nucleotide variants listed in Table 1.

The lower portion of Table 2 shows the standard Gibbs free energy of reaction ΔG° for each binding/displacement reaction based on the G° listed in the upper portion of Table 2. As shown, ΔG° is more negative for the desired binding/displacement reaction involving the target oligonucleotide, miRNA-375, and, thus, more thermodynamically favorable, than for the competing reactions involving the single-nucleotide variants (MM₁, MM₅, MM₁₂, MM₁₈, and MM₂₂). Nonetheless, competing reactions involving single-nucleotide variants binding to the probe oligonucleotide can still occur under these conditions. To improve the selectivity toward the target oligonucleotide, miRNA-375, an excess amount of the protector oligonucleotide can be added to the assay medium. According to Le Chatelier's principle, the excess amount of protector oligonucleotide will make the Gibbs free energy of reaction (ΔG) more positive than the ΔG° value shown in Table 2 for each of these reactions. To provide an optimal level of selectivity for the target oligonucleotide (T) over the single-nucleotide variants, the excess amount of protector oligonucleotide (P) can be selected so as to make ΔG≈0 for the desired reaction: T+PC [symbol] TC+P. The concentration of P needed to achieve this can be determined by solving the following equation, where [P] is the concentration of protector oligonucleotide, [C] is the concentration of probe oligonucleotide, R is the ideal gas constant, and τ is the temperature in Kelvins:

ΔG=ΔG°+ln([P]/[C])/Rτ. (1)

In this case, the concentration ratio [P]/[C]=9.93 results in ΔG=0 for the desired reaction.

It is also possible to select the concentration of P to provide an optimal level of selectivity for T over a specific single-nucleotide variant (SNV). To achieve this, the concentration of P is selected using equation (1) to make ΔG for the desired reaction to be equal to −ΔΔG°/2, where ΔΔG°=ΔG°_{SNV}−ΔG°_{T}. Table 3 below lists the concentration ratio [P]/[C] that provides this optimal level of selectivity against each of the SNVs (MM₁, MM₅, MM₁₂, MM₁₈, and MM₂₂).

TABLE 3

| SNV | ΔG°_{SNV} (kcal/mol) | ΔΔG° (kcal/mol) | [P]/[C] |
|---|---|---|---|
| MM₁ | −0.37 | 0.99 | 4.31 |
| MM₅ | 2.21 | 3.57 | 0.48 |
| MM₁₂ | 4.35 | 5.71 | 0.08 |
| MM₁₈ | 3.26 | 4.62 | 0.20 |
| MM₂₂ | −0.37 | 0.99 | 4.31 |

g. Experimental Results

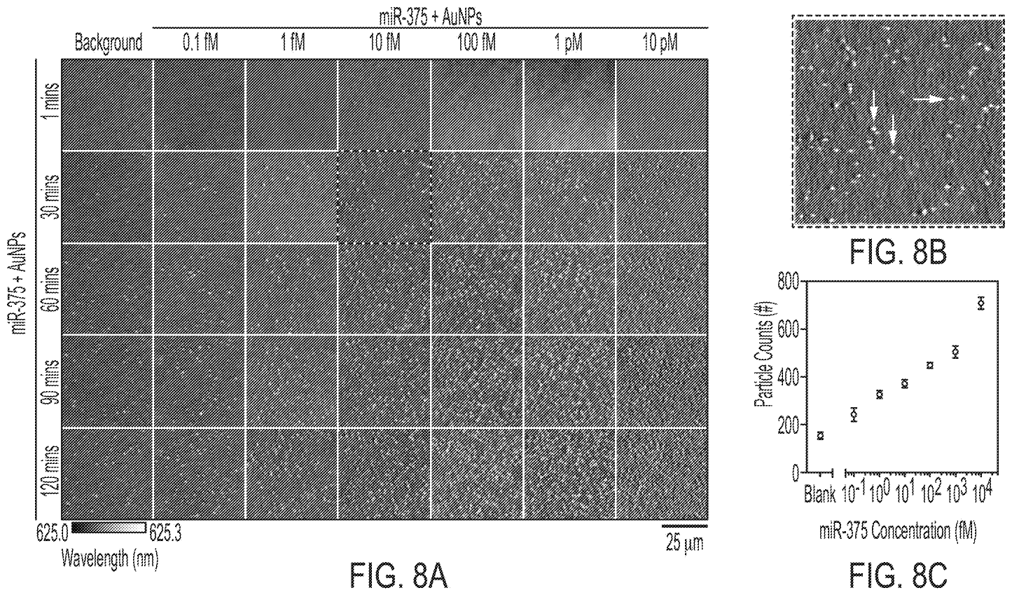

FIG. 8A-8C illustrate the results of experiments that were performed to detect miRNA-375 over a wide range of concentrations. The experiments were performed by mixing a constant amount of the nanoparticle probe (AuNP) with a defined concentration of miRNA in a PDMS well (about 10 μL/well) in which a PC functionalized with the capture oligonucleotide was adhered to the bottom of the well. Serial dilutions were used to provide defined concentrations of miRNA-375 ranging from 100 aM (0.1 fM) to 10 μM (104 fM). Immediately following the introduction of miR-375, a 50×50 μm² area of the PC surface is scanned at 30-minute intervals for up to 2 hours. Peak wavelength grey-scale images of the scans of the PC surface for each of the miRNA-375 concentrations, and for a background no miRNA-375 control, taken at 1 minute, 30 minutes, 60 minutes, 90 minutes, and 120 minutes after introduction are shown in FIG. 8A. The increasing number of PC-bound particles over time may be interpreted as resulting from the coupled kinetic dependence of the binding/displacement reaction and the surface capture reaction.

The grey-scale image for 10 fM at 30 minutes is shown in FIG. 8B in an expanded form with arrows identifying representative instances of single nanoparticle probes bound to the surface of the PC. Thus, the PC-bound nanoparticle probes were resolvable at single particle digital resolution. To count the PC-bound nanoparticle probes over time, the counting algorithm discussed below was used. The PC-bound particle count after 2 hours for each of the miRNA-375 concentrations (averaged over 3 independent experiments) is shown in FIG. 8C. The “blank” represents the no miRNA-375 control. The error bars represent the standard errors. These results show that the PRAM technique of counting individual nanoparticle probes bound the PC surface was able to distinguish between different miRNA-375 concentrations ranging from 100 aM (0.1 fM) to 10 pM (104 fM).

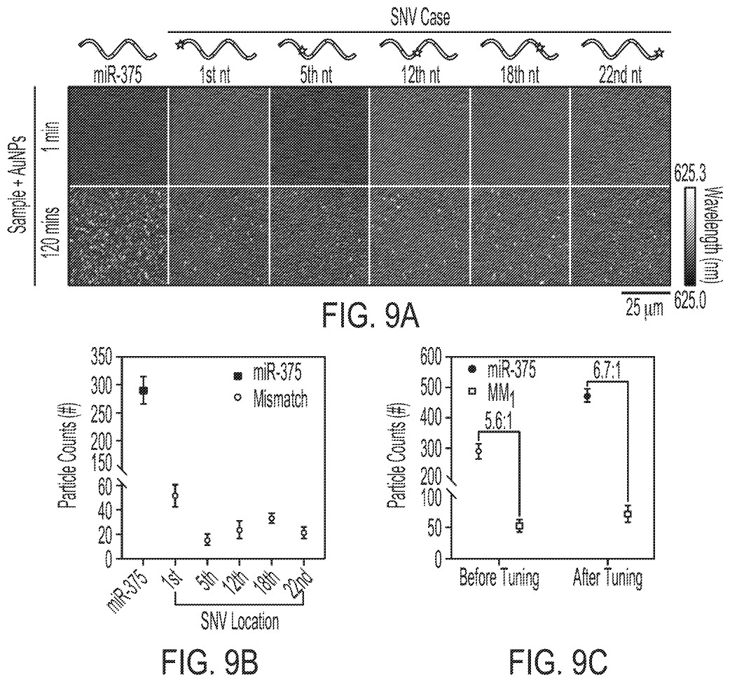

To test for selectivity, the PRAM assay was performed using the five different SNVs (MM₁, MM₅, MM₁₂, MM₁₈, and MM₂₂) at a concentration of 1 pM instead of miRNA-375, and these results were compared to a PRAM assay performed using miRNA-375 at the same concentration. FIGS. 9A-9C illustrate the results. Peak wavelength grey-scale images for the scans of the PC surface for the miRNA-375 and for each of the SNVs, taken at 1 minute and 120 minutes after introduction, are shown in FIG. 9A. Shown in FIG. 9B are the counts of PC-bound nanoparticle probes for miRNA-375 and for each of the SNVs averaged over 3 independent experiments (the error bars represent the standard errors). These results show a 83% to 94% reduction in the number of counts after 2 hours for the SNVs as compared to miRNA-375.

Although an 83% reduction in the competing reaction for MM₁ may be considered acceptable, tuning the amount of protector oligonucleotide can provide a further reduction. As discussed above, optimal selectivity against the MM₁ reaction can be achieved by making ΔG for the desired miRNA-375 reaction to be equal to −ΔΔG°/2 according to equation (1), where ΔΔG° is the difference between ΔG° for the MM₁ reaction and ΔG° for the desired miRNA-375 reaction. The optimal [P]/[C] ratio for this case is 4.31, as shown above in Table 3. When this optimal [P]/[C] ratio was used, the discrimination against the MM₁ reaction was found to increase from about 5.6-fold (before tuning) to about 6.7-fold (after tuning), as illustrated in FIG. 9C. The amount of excess protector oligonucleotide is lower than the amount of excess protector oligonucleotide used in the before-tuning (ΔG≈0) strategy, thereby making both the miRNA-375 reaction and the MM₁ reaction more favorable. This is reflected by the “after tuning” results having a higher number of particle counts than the “before tuning” results for both miRNA-375 and MM₁, as shown in FIG. 9C. It appears that this particle count increase resulted in saturation of the PC surface after 2 hours, thereby limiting the discrimination ratio improvement.

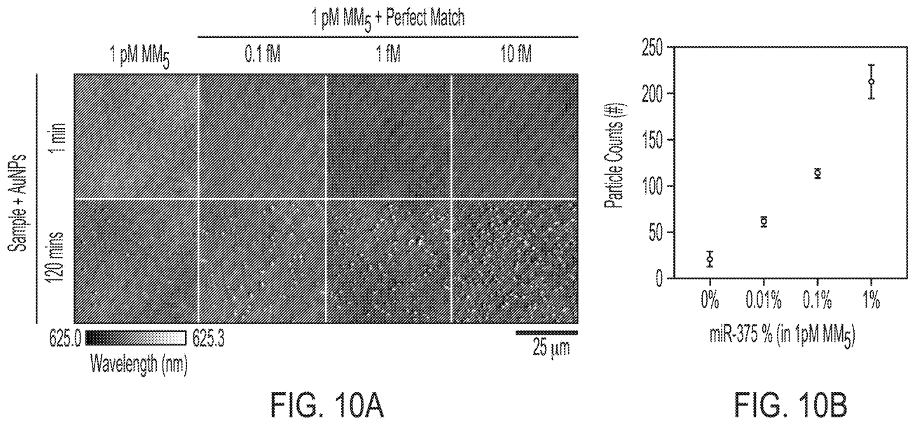

To further test the selectivity of the nanoparticle probe for miRNA-375, experiments were performed using various concentrations of miRNA-375 (100 aM, 1 fM, 10 fM) in a much higher concentration (1 pM) of one of the SNVs (MM₅ was used here). The results are shown in FIGS. 10A and 10B. Despite the relatively high mismatch background, increasing counts of PC-bound nanoparticle probes were observed as a function of increasing miRNA-375 concentration and assay time. Shown in FIG. 10A are peak wavelength grey-scale images for the scans of the PC surface for 1 pM MM₅ without miRNA-375 and with the three different concentrations miRNA-375, taken at 1 minute and 120 minutes after introduction. Shown in FIG. 10B are the particle counts as a function of the concentration ratio [miRNA-375]/[MM₅]. Each data point represents the average of 3 independent experiments, and the error bars represent the standard errors. The overall particle counts are lower than the results shown in FIG. 8C. This implies that the mismatch MM₅ alters the miRNA-375 kinetics by non-specific binding. In addition to potential non-specific binding of the capture oligonucleotide, the mismatch MM₅ may transiently bind to the probe oligonucleotide. However, as evidenced by the increase in counts as the miRNA-375 concentration increases, instances of spuriously-bound MM₅ are expected to be displaced by miRNA-375.

### 6. Processing and Counting Algorithm for PRAM Images

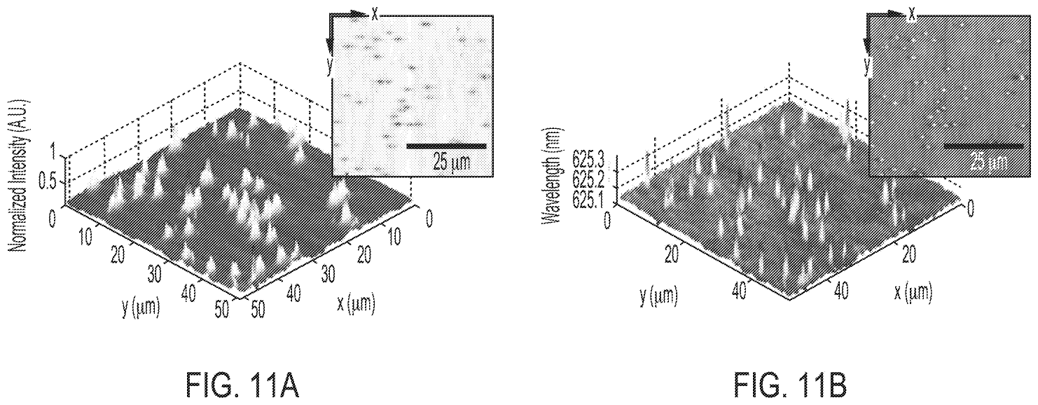

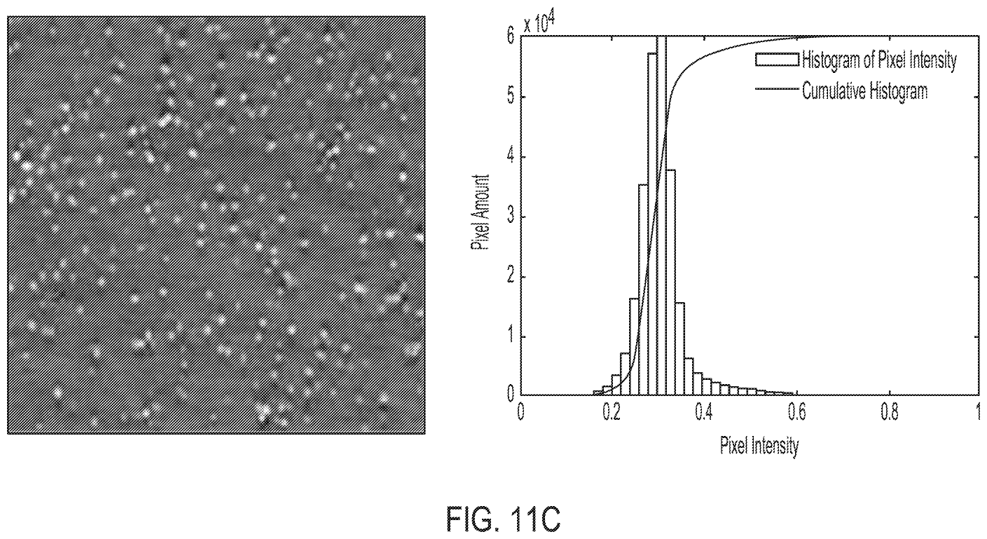

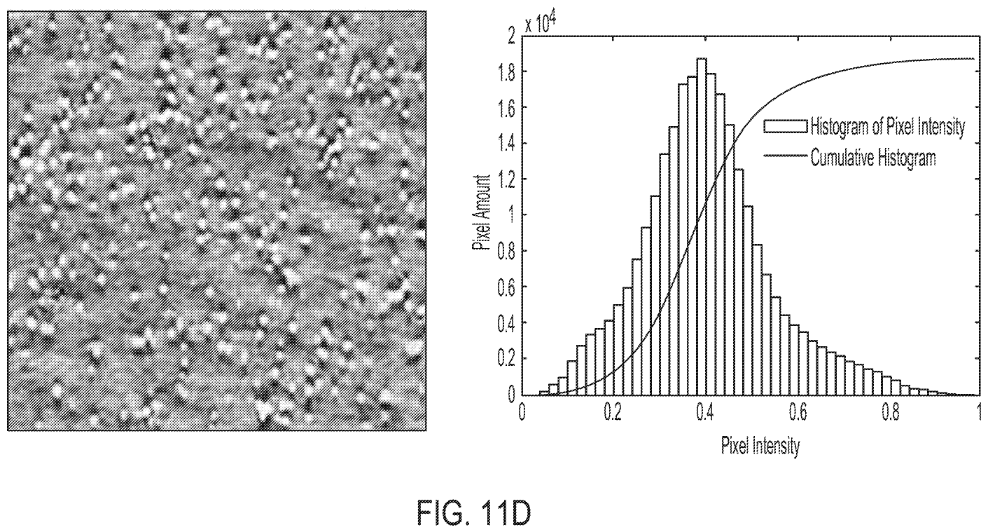

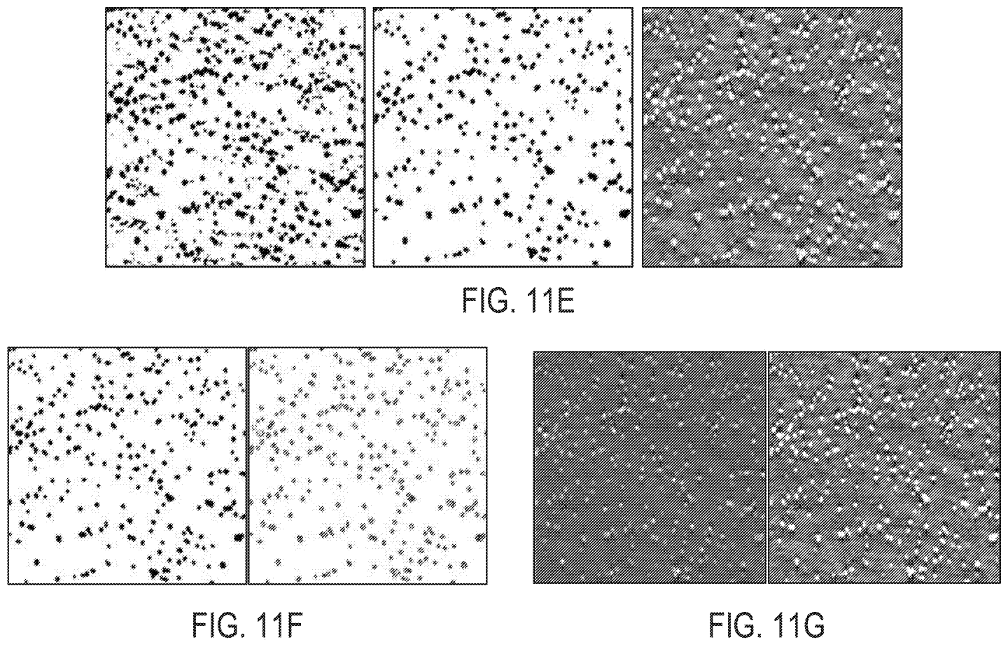

FIGS. 11A-11G illustrate aspects of a processing and counting algorithm for PRAM-acquired images. Shown in FIG. 11A is an example greyscale PIV image of nanoparticle probes on the PC surface and a 3D contour plot showing the corresponding normalized intensity of each pixel in the field of view. Shown in FIG. 11B is an example greyscale PWV image of nanoparticle probes on the PC and a 3D contour plot showing the corresponding peak reflected wavelengths of each pixel in the field of view. It was found that the nanoparticle probes exhibit sharper features in the PWV images than in the PIV images. Therefore, the PWV images were used for counting the PC-bound nanoparticle probes in the studies reported herein.

As the first step, a Contrast Limited Adaptive Histogram Equalization (CLAHE) algorithm was applied to normalize the contrast of the PWV images. The PWV images and corresponding pixel intensity histograms before and after applying CLAHE are shown in FIG. 11C and FIG. 11D, respectively. The normalized image is then Wiener filtered, followed by a simple binarization with a threshold of half the maximum pixel intensity as a rudimentary segmentation, resulting in the example image shown in the left panel of FIG. 11E. To remove background noise, dilation and erosion is then applied to the binarized image, resulting in the example image shown in the middle panel in FIG. 11E. An example of the resulting overlapped image is shown in the right panel in FIG. 11E. To eliminate the inaccuracy caused by nanoparticle clusters, the local maxima within each segmented pattern are used to indicate individual nanoparticles, as shown in FIG. 11F. Then, a watershed algorithm is used to determine the number of detected nanoparticles presented in the image, as shown in FIG. 11G.

### 7. On-Chip Toehold-Mediated Bridge Assay for Single Strand Nucleic Acid Detection

In an alternative approach, a PC surface that is functionalized with probes is first exposed to the target analyte, so as to allow the target analyte to bind to the probes on the PC surface, and then exposed to conjugated nanoparticles that include a metallic nanoparticle conjugated to a reporter. The conjugated nanoparticles bind to target-activated probes (probes that have bound to the target analyte) to form nanoparticle probes on the PC surface that can be individually detected and counted using PRAM.

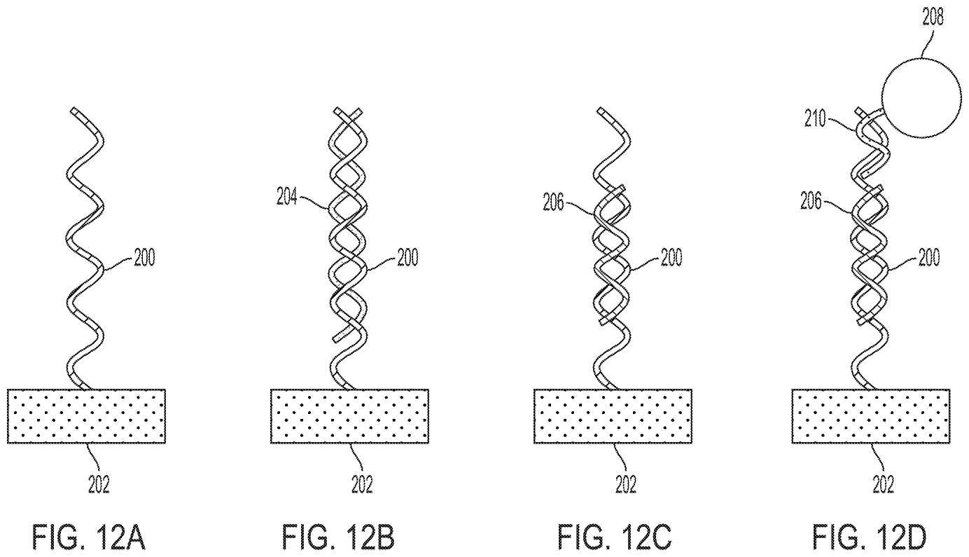

For a target analyte that is an oligonucleotide (e.g., a miRNA), the probe can be a probe oligonucleotide that is bound to the PC surface, and the reporter in the conjugated nanoparticles can be a reporter oligonucleotide. The probe oligonucleotide includes a first portion that is complementary to the target oligonucleotide and a second portion that is complementary to at least part of the reporter oligonucleotide. FIGS. 12A-12D schematically illustrate steps of an example assay. Initially, a plurality of probe oligonucleotide probes are bound to the surface of a PC, as exemplified in FIG. 12A by a probe oligonucleotide 200 bound to a PC 202. A protector oligonucleotide is then added to block the probe oligonucleotides on the PC surface. Specifically, the protector oligonucleotide binds to at least part of the first portion of the probe oligonucleotide and at least part of the second portion of the probe oligonucleotide. FIG. 12B shows the probe oligonucleotide 200 bound to a protector oligonucleotide 204.

When the functionalized PC surface is exposed to the target oligonucleotide (e.g., in a sample), the target oligonucleotide binds to the probe oligonucleotide and displaces the protector oligonucleotide. An excess amount of the protector oligonucleotide can be provided so as to make binding of the target oligonucleotide more thermodynamically favorable than binding of SNVs. FIG. 12C shows the probe oligonucleotide 200 bound to a target oligonucleotide 206 that has displaced the protector oligonucleotide 204. Displacement of the protector oligonucleotide caused by binding of the target oligonucleotide exposes the second portion of the probe oligonucleotide, which enables the reporter oligonucleotide of a conjugated nanoparticle to bind to the probe oligonucleotide. FIG. 12D shows a gold nanoparticle 208 conjugated to a reporter oligonucleotide 210 that has bound to the probe oligonucleotide 200.

In an experimental example, single-strand DNA probe molecules (28 bps) modified with amine groups were covalently immobilized on an epoxy silane terminated PC surface. Predesigned complementary single-strand DNA molecules (21 bps) were added and incubated (4 C degree for 2 hours) to block the probes via DNA hybridization. Afterwards, single-strand target DNA molecules (15 bps) were added and incubated at 4 C degree for 4 hours to induce the strand replacement via toe-hold exchange reaction, which results in the top region of the probes (10 bps) exposed and unhybridized. An NHS-activated gold NanoUrchin conjugation kit (Cytodiagnostics, Burlington, Ontario) was used for conjugating gold nanoparticles with NeutrAvidin proteins (Thermo Scientific). Biotinylated single strand reporter DNA molecules (10 bps) were then incubated with the avidin-coated nanoparticles at room temperature for 2 hours to form reporter-conjugated nanoparticles via biotin-avidin reaction. The prepared reporter-conjugated nanoparticles were immediately introduced to the PC surface so as to bind (bridge) to target-activated probes on the PC surface. The target-activated probes that were bridged with the nanoparticles could then be individually detected and counted using PRAM.

### 8. Conclusion

The experimental results presented herein demonstrate that by integrating principled DNA nanotechnology with PC biosensors, highly selective and sensitive diagnostics are achievable. Each miRNA target molecule translates into a digitally observable nanoparticle probe that is attached to the PC, via two highly specific biomolecular recognition events. In one approach, binding of the miRNA to the probe oligonucleotide in the nanoparticle probe is followed by binding of the probe oligonucleotide to the capture oligonucleotide on the PC surface. In another approach, binding of the miRNA to the probe oligonucleotide on the PC surface is followed by binding of functionalized nanoparticles to target-activated probes. The assays can be conducted at room temperature and without any target amplification or wash steps. Single-nucleotide mismatches can be located across the candidate miRNA when using a DNA probe/protector system that is free energy tuned.

The digital resolution capability of the PRAM biosensor microscopy allows for direct and dynamic signal accumulation, thereby enabling rapid miRNA detection. Given the simplicity of the assay and the commercial availability of the reagents involved (with low cost), it is expected that the PRAM method can be applied to detect DNA, proteins, and small molecules as well. Lastly, the PC-mediated enhanced absorption can achieve digital detection of nanoparticle probes, which it is expected can be implemented in a low-cost and portable point of care device.

What is claimed is:

1. An assay medium, comprising:
a buffer solution;
a plurality of nanoparticle probes in the buffer solution, wherein the nanoparticle probes comprise metallic nanoparticles in which each metallic nanoparticle is conjugated to a probe oligonucleotide with a protector oligonucleotide bound to the probe oligonucleotide, wherein a first portion of the probe oligonucleotide is complementary to a target oligonucleotide such that the target oligonucleotide is able to bind to the probe oligonucleotide and displace the protector oligonucleotide therefrom; and
an excess amount of the protector oligonucleotide in the buffer solution.

2. The assay medium of claim 1, further comprising:
a substrate; and
a plurality of capture oligonucleotides conjugated to the substrate, wherein at least a portion of each capture oligonucleotide is complementary to a second portion of the probe oligonucleotide.

3. The assay medium of claim 2, wherein the protector oligonucleotide is bound to (i) at least part of the first portion of the probe oligonucleotide and (ii) at least a part of the second portion of the probe oligonucleotide.

4. The assay medium of claim 3, wherein displacement of the protector oligonucleotide from the probe oligonucleotide by the target oligonucleotide exposes the second portion of the probe oligonucleotide such that the capture oligonucleotide is able to bind to the probe oligonucleotide.

5. The assay medium of claim 2, wherein the substrate comprises a photonic crystal.

6. The assay medium of claim 5, wherein the metallic nanoparticles have a spiked surface.

7. The assay medium of claim 5, wherein the metallic nanoparticles are nano-urchins.

8. The assay medium of claim 5, wherein the metallic nanoparticles are gold nanoparticles.

9. The assay medium of claim 5, wherein the metallic nanoparticles have a surface plasmon resonance at a wavelength that matches a resonant wavelength of the photonic crystal.

10. The assay medium of claim 5, wherein the metallic nanoparticles are magnetic.

11. The assay medium of claim 5, wherein the metallic nanoparticles have a diameter that is between about 50 nanometers and about 100 nanometers.

12. The assay medium of claim 1, wherein the excess amount of the protector oligonucleotide in the buffer solution is such that binding of the target oligonucleotide to the probe oligonucleotide with displacement of the protector oligonucleotide therefrom has a reaction free energy (ΔG) that is zero or negative.

13. The assay medium of claim 12, wherein the excess amount of the protector oligonucleotide in the buffer solution provides selectivity over a plurality of different single-nucleotide variants (SNVs) of the target oligonucleotide in that binding of each SNV to the probe oligonucleotide with displacement of the protector oligonucleotide therefrom has an associated reaction free energy (ΔG) that is positive.

14. A method, comprising:
exposing a surface of a photonic crystal to:
a sample comprising a target oligonucleotide;
a plurality of nanoparticle probes configured to bind to the target oligonucleotide, wherein binding of the target oligonucleotide to a given nanoparticle probe displaces a protector oligonucleotide therefrom and enables the given nanoparticle probe to bind to the surface of the photonic crystal; and
an excess amount of the protector oligonucleotide;
determining a number of nanoparticle probes that have bound to the surface of the photonic crystal; and
correlating the number of nanoparticle probes that have bound to the surface of the photonic crystal with an abundance of the target oligonucleotide in the sample.

15. The method of claim 14, wherein the nanoparticle probes comprise metallic nanoparticles in which each metallic nanoparticle is conjugated to a probe oligonucleotide with the protector oligonucleotide bound to the probe oligonucleotide, wherein a first portion of the probe oligonucleotide is complementary to the target oligonucleotide such that the target oligonucleotide is able to bind to the probe oligonucleotide and displace the protector oligonucleotide therefrom.

16. The method of claim 15, wherein the surface of the photonic crystal is conjugated to a plurality of capture oligonucleotides, wherein at least a portion of each capture oligonucleotide is complementary to a second portion of the probe oligonucleotide.

17. The method of claim 16, wherein the protector oligonucleotide is bound to (i) at least part of the first portion of the probe oligonucleotide and (ii) at least part of the second portion of the probe oligonucleotide.

18. The method of claim 17, wherein displacement of the protector oligonucleotide from the probe oligonucleotide by the target oligonucleotide exposes the second portion of the probe oligonucleotide such that the capture oligonucleotide is able to bind to the probe oligonucleotide.

19. The method of claim 14, wherein the photonic crystal is configured to reflect light with a peak intensity at a resonant wavelength, wherein binding of one of the nanoparticle probes to a location on the surface of photonic crystal increases the resonant wavelength of reflected light at that location as compared to unbound locations and reduces the peak reflected intensity as compared to unbound locations.

20. The method of claim 19, wherein determining the number of nanoparticle probes that have bound to the surface of the photonic crystal comprises:
illuminating the surface of the photonic crystal with incident light;
receiving reflected light that has reflected from each of a plurality of locations on the surface of the photonic crystal in response to illumination by the incident light; and
detecting at least one of (i) an increased resonant wavelength in the reflected light from one or more of the plurality of locations or (ii) a reduced reflected peak intensity in the reflected light from one or more of the plurality of locations.

21. A method, comprising:
providing a functionalized photonic crystal, wherein the functionalized photonic crystal comprises a plurality of probe oligonucleotides bound to a surface of the photonic crystal, wherein each probe oligonucleotide is bound to a protector oligonucleotide and includes a first portion that is complementary to a target oligonucleotide such that the target oligonucleotide is able to bind to the probe oligonucleotide and displace the protector oligonucleotide therefrom, and wherein the probe oligonucleotide further includes a second portion that is exposed when the target oligonucleotide displaces the protector oligonucleotide;
exposing the functionalized photonic crystal to (i) a sample comprising the target oligonucleotide and (ii) conjugated nanoparticles, wherein each conjugated nanoparticle comprises a metallic nanoparticle conjugated to a reporter oligonucleotide that is configured to bind to the second portion of the probe oligonucleotide so as to form an individual nanoparticle probe bound to the surface of the photonic crystal;
determining a number of nanoparticle probes bound to the surface of the photonic crystal; and
correlating the number of nanoparticle probes bound to the surface of the photonic crystal with an abundance of the target oligonucleotide in the sample.

22. The method of claim 21, further comprising:
exposing the functionalized photonic crystal to an excess amount of the protector oligonucleotide.

23. The method of claim 21, wherein the second portion of the probe oligonucleotide is complementary to at least part of the reporter oligonucleotide.

24. The method of claim 21, wherein the protector oligonucleotide is bound to (i) at least part of the first portion of the probe oligonucleotide and (ii) at least part of the second portion of the probe oligonucleotide.

25. The method of claim 21, wherein the photonic crystal is configured to reflect light with a peak intensity at a resonant wavelength, wherein a location on the surface of photonic crystal at which one of the nanoparticle probes is bound has an increased resonant wavelength of reflected light at that location as compared to unbound locations and a reduced peak reflected intensity as compared to unbound locations.

26. The method of claim 25, wherein determining the number of nanoparticle probes bound to the surface of the photonic crystal comprises:
illuminating the surface of the photonic crystal with incident light;
receiving reflected light that has reflected from each of a plurality of locations on the surface of the photonic crystal in response to illumination by the incident light; and
detecting at least one of (i) an increased resonant wavelength in the reflected light from one or more of the plurality of locations or (ii) a reduced reflected peak intensity in the reflected light from one or more of the plurality of locations.
